(19) United States
(12) Patent Application Publication
(10) Pub. No.: US 2015/0029512 A1
(43) Pub. Date: Jan. 29, 2015

(54) SURFACE EMITTING LASER AND OPTICAL APPARATUS

(71) Applicant: CANON KABUSHIKI KAISHA, Tokyo (JP)

(72) Inventors: Tatsuro Uchida, Machida-shi (JP); Mamoru Uchida, Yokohama-shi (JP)

(21) Appl. No.: 14/333,196

(22) Filed: Jul. 16, 2014

(30) Foreign Application Priority Data
Jul. 24, 2013 (JP) 2013-153784

(51) Int. Cl. A61B 3/10; A61B 3/12; G01N 21/47; H01S 3/00; G01B 9/02

(52) U.S. Cl. CPC A61B 3/102; H01S 3/005; G01B 9/02091; G01N 21/4795; A61B 3/12; G01N 2201/06113
USPC 356/479; 372/107; 351/206

(57) ABSTRACT

A surface emitting laser comprises an upper reflecting mirror, a lower reflecting mirror, and an active layer located between those mirrors. A cavity of the surface emitting laser consists of the upper reflecting mirror and the lower reflecting mirror. In the surface emitting laser, a plurality of individual light receiving portions is located in an optical path inside the cavity in order to detect a laser beam.

### BACKGROUND OF THE INVENTION

1. Field of the Invention

The present invention relates to a surface emitting laser and an optical coherence tomography.

2. Description of the Related Art

An example of a surface emitting laser includes a vertical cavity surface emitting laser (hereinafter referred to as “VCSEL”). In the VCSEL, the top and bottom of an active layer are sandwiched with two reflecting mirrors, and a cavity is located in a direction vertical to a surface of a substrate. From the VCSEL, a laser beam is emitted in the direction vertical to the surface of the substrate. Another example of the surface emitting laser includes a variable wavelength VCSEL in which a wavelength of light to be emitted is variable. An example of such a variable wavelength VCSEL includes a laser in which a gap is provided between an upper reflecting mirror and an active layer. Herein, a cavity length can be changed by moving the upper reflecting mirror in a direction of an optical path of a laser beam so that a wavelength of light to be emitted can be changed.

Herein, in cases where the VCSEL is used as an optical source for an optical coherence tomography or that of a laser beam printer, it is preferable to make intensity of the light to be emitted fall within a predetermined range so as not to fluctuate as much as possible. Further, a transverse mode may preferably be a single-mode.

Japanese Patent Application Laid-Open No. 2007-13227 (hereinafter called “Patent Literature 1”) discloses a surface emitting type semiconductor laser as the surface emitting laser. Herein, a light absorbing layer 1 provided in an optical waveguide inside the laser as shown in FIG. 9B is used as a light receiving portion, and light output of a laser beam is monitored. FIG. 9A is a top view, while FIG. 9B is a cross-sectional view. Herein, an active region 15, DBR mirrors 13 and 17 included in a cavity, and electrodes 21, 22, and 23 are shown. This surface emitting type semiconductor laser has a cylindrical shape as shown in FIG. 9A, and has one light absorbing layer (light receiving portion). By using this light absorbing layer (light receiving portion), it is possible to detect that the transverse mode state of the light to be emitted has changed from the single-mode to a multimode.

### SUMMARY OF THE INVENTION

A surface emitting laser according to an embodiment of the present invention comprises an upper reflecting mirror, a lower reflecting mirror, and an active layer located between those mirrors, the surface emitting laser further consisting of a plurality of individual light receiving portions located in an optical path inside a cavity consisting of the upper reflecting mirror and the lower reflecting mirror in order to detect a laser beam.

Further features of the present invention will become apparent from the following description of exemplary embodiments (with reference to the attached drawings).

### BRIEF DESCRIPTION OF THE DRAWINGS

FIG. 1A is a top view of an element of a surface emitting laser according to a first embodiment of the present invention, while FIG. 1B is a cross-sectional view taken along line A-A′ in FIG. 1A.

FIGS. 2A to 2D are graphs for explaining a mechanism that detects intensity of light output and an operating status of a transverse mode in the surface emitting laser according to the embodiment of the present invention.

FIG. 3A is a top view of an element of a surface emitting laser according to a second embodiment of the present invention, while FIG. 3B is a cross-sectional view taken along line B-B′ in FIG. 3A.

FIGS. 4A to 4D are views for explaining structures in which light receiving portions, according to the embodiment of the present invention, are located at different positions, respectively.

FIG. 5 is a view for explaining the light receiving portion according to the embodiment of the present invention.

FIG. 6 is a view for explaining an optical coherence tomography according to the embodiment of the present invention.

FIG. 7A is a top view of an element of a surface emitting laser according to Example 1 of the present invention, while FIG. 7B is a cross-sectional view taken along line C-C′ in FIG. 7A.

FIG. 8 is a view showing a vicinity of the light receiving portion of the surface emitting laser L according to Example 1 of the present invention.

FIG. 9A is a top view showing a structure of a surface emitting type semiconductor laser in the related art, while FIG. 9B is a cross-sectional view of the same.

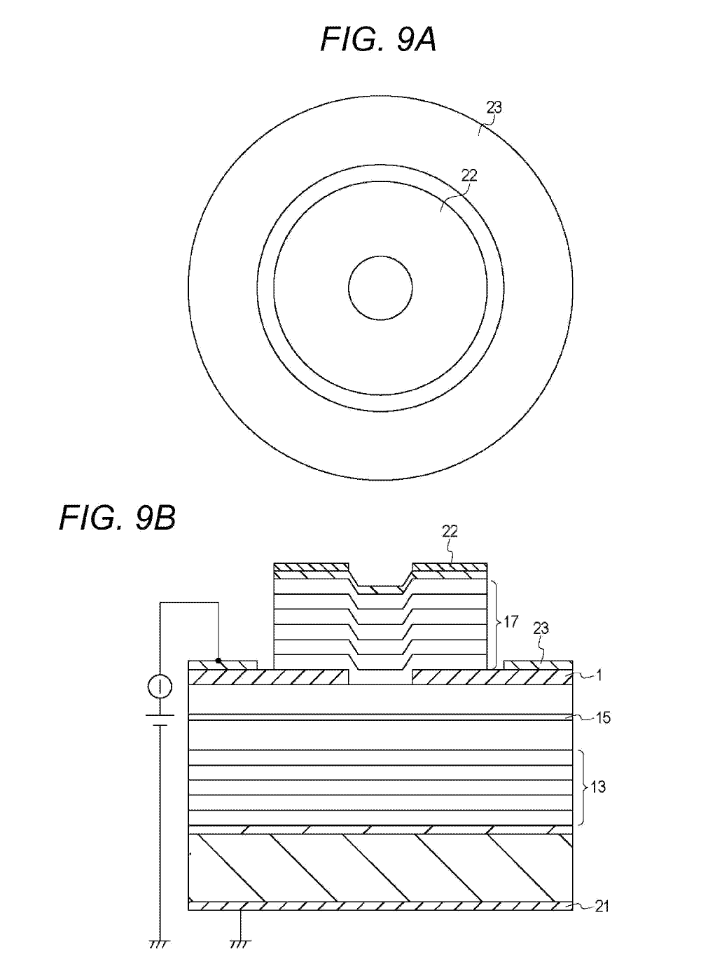

### DESCRIPTION OF THE EMBODIMENTS

Herein, embodiments of the present invention will be described.

### First Embodiment

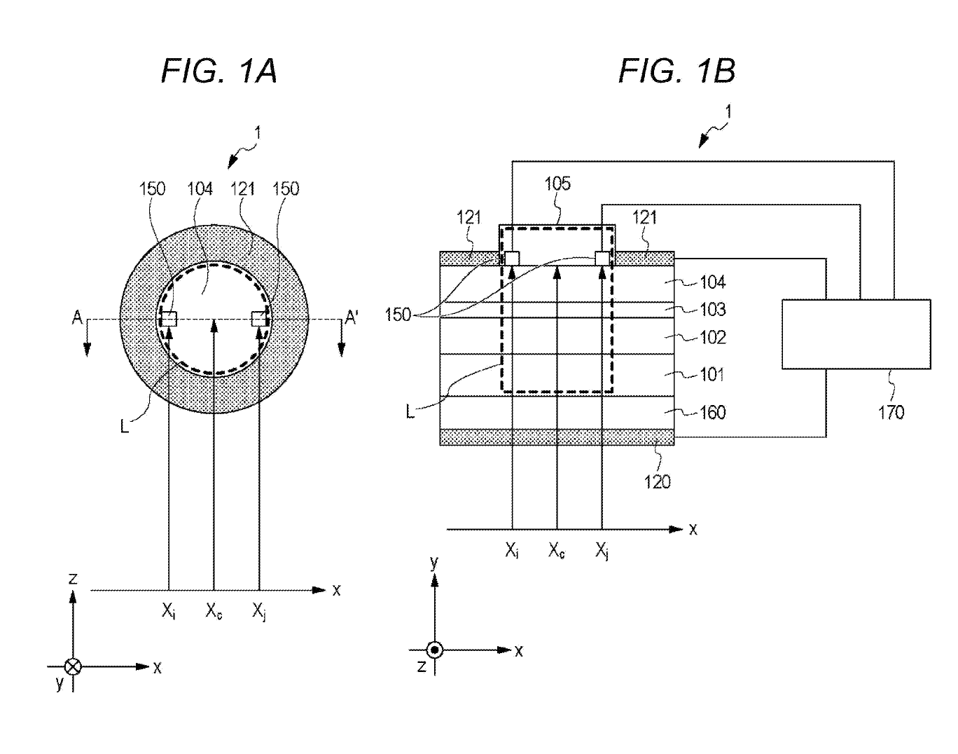

A surface emitting laser according to a first embodiment will be described. FIG. 1A is a top view of an element of the surface emitting laser according to the present embodiment, while FIG. 1B is a cross-sectional view taken along line A-A′ in FIG. 1A. FIG. 1A herein is the view showing the element without an upper reflecting mirror 105.

A surface emitting laser 1 comprises a lower reflecting mirror 101, a first cladding layer 102, an active layer 103, a second cladding layer 104, and a disk-shaped upper reflecting mirror 105 on the top of a substrate 160. The first cladding layer 102 is located on the lower reflecting mirror 101. The active layer 103 is located on the first cladding layer 102. The second cladding layer 104 is located on the active layer 103. Finally, the upper reflecting mirror 105 is located on the second cladding layer 104. Under the substrate 160, there is formed a first electrode 120. On the other hand, a second electrode 121 in a ring-shape is formed on the second cladding layer 104. When current is applied to the active layer 103 with the first and second electrodes 120, 121, light is emitted in the active layer 103. The emitted light makes a round trip inside a cavity consisting of the lower reflecting mirror 101 and upper reflecting mirror 105. This results in stimulated emission. The stimulated emission light inside the cavity passes through the upper reflecting mirror 105 in a direction vertical to a surface of the substrate 160, or in a y-direction. Then, the light is emitted as a laser beam having a specific wavelength. The surface emitting laser according to the present embodiment consists of a plurality of light receiving portions 150 for detecting a transverse mode of the laser beam. The light receiving portions herein are located in an optical path inside the cavity consisting of the upper reflecting mirror 105 and the lower reflecting mirror 101. Herein, the optical path inside the cavity represents a region through which light passes when the light resonates within the cavity consisting of the lower reflecting mirror 101 and the upper reflecting mirror 105. In FIGS. 1A and 1B, the optical path inside the cavity is shown as a region L (shown with dotted lines) inside the cavity which consisting of the upper reflecting mirror 105 and lower reflecting mirror 101.

The surface emitting laser 1 in FIGS. 1A and 1B has a pair of light receiving portions 150 on the top of the second cladding layer 104. Further, in FIGS. 1A and 1B, positions where the light receiving portions 150 are located in an x-direction, which is parallel to the surface of the substrate 160, are represented as Xᵢ and Xⱼ. In the present embodiment, Xᵢ and Xⱼ are located inside a circular region encircled by the ring-shaped electrode 121 which is located on the cladding layer 104. The center of the circular region represented as X_{c} is located between Xᵢ and Xⱼ, and Xᵢ and Xⱼ are located at symmetrical positions equidistant from the center X_{c}.

A control unit 170 consists of a driving circuit and a measuring circuit. The driving circuit injects a current (or applies a voltage) between the electrodes 121, 120 in order to drive the surface emitting laser 1. The measuring circuit individually receives photoelectric conversion signals transmitted from the pair of light receiving portions 150, and measures light intensity of each signal. The control unit 170 can further change driving current of the surface emitting laser 1 based on each detected photoelectric conversion signal transmitted from the pair of light receiving portions 150.

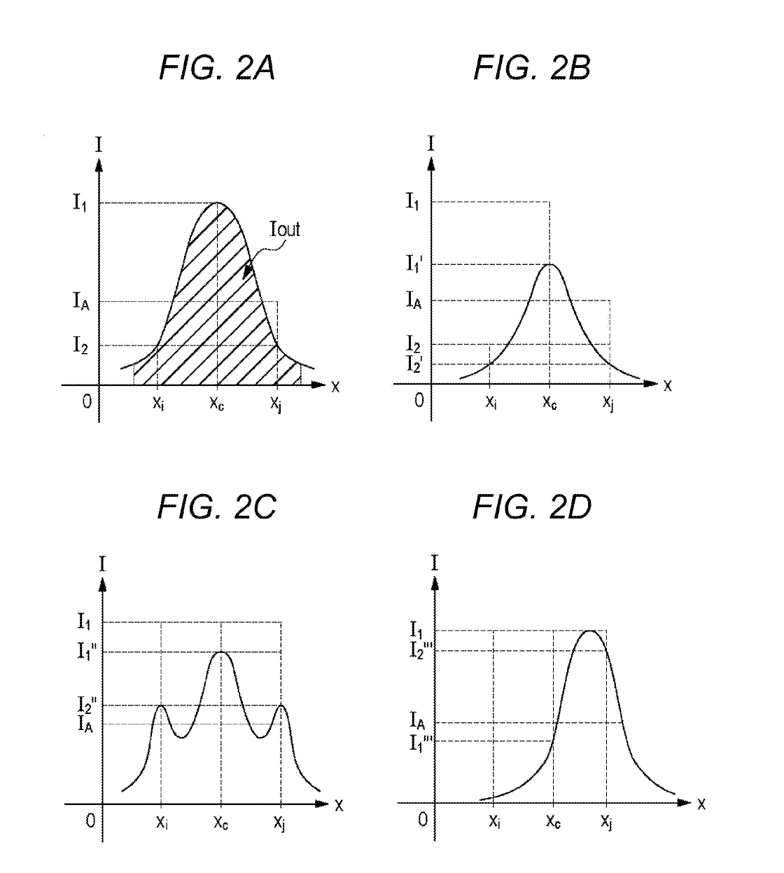

Next, a method for determining intensity of light output and an operating status of the transverse mode by using the surface emitting laser according to the present embodiment will be described with reference to FIGS. 2A to 2D. The abscissa of each graph in FIG. 2A to 2D represents positions in the x-direction shown in FIGS. 1A and 1B, while the ordinate of each graph represents light intensity of a laser beam. Herein, the intensity of the light output of the laser beam represents intensity (quantity) of light to be eventually emitted from the surface emitting laser according to the present embodiment. In other words, the intensity of the light output is an integrated value of intensity distribution of a laser beam to be emitted. For example, the intensity of the light output is represented as Iₒᵤₜ shown with diagonal lines in a graph of light-intensity distribution in FIG. 2A.

FIG. 2A is a graph showing the light-intensity distribution in a case where a laser oscillates in a single-mode and desired light intensity is obtained at the center X_{c}. On the other hand, FIG. 2B is a graph showing the light-intensity distribution in a case where a laser oscillates in the single-mode operation but the desired light intensity cannot be obtained at the center X_{c} due to deterioration of the light intensity. Further, FIG. 2C is a graph showing a case where a laser oscillates in a multimode, not in the single-mode. Finally, FIG. 2D is a graph showing the light-intensity distribution in a case where a laser oscillates in the single-mode, but a peak position of the light intensity is shifted from the center X_{c}. These are typical light-intensity distributions of the surface emitting laser.

According to the present embodiment, the light-intensity distribution is estimated by detecting a plurality of optical signals at predetermined positions with the plurality of light receiving portions. Based on the estimated result, it is possible to determine whether the transverse mode state of the laser beam is the single-mode or the multimode. According to the present embodiment, based on the estimated result, it is further possible to measure the intensity of the light output and to detect whether necessary light intensity is obtained at a necessary position. In this manner, changing of current to be injected to the surface emitting laser is controlled as required based on the operating status of the surface emitting laser.

Hereinafter, the above-mentioned description will be described in detail.

In a case where a laser oscillates in the light-intensity distribution as shown in FIG. 2A, light intensity which is to be detected by the light receiving portion located at either Xᵢ or Xⱼ is represented as I₂. In a case where the value of I₂ is smaller than that of I_{A}, it is determined in the present embodiment that the laser oscillates in the single-mode. In a case where light intensity smaller than I_{A} is detected at any one of Xᵢ or Xⱼ, it is also determined that the laser oscillates in the single-mode. On the other hand, in a case where light intensity larger than I_{A} is detected at both Xᵢ and Xⱼ, it is determined that the laser oscillates in the multimode.

Seeing the value of I_{A}, it can be determined whether the laser oscillates in the single-mode. For example, the value can be preset by determining a relation between an amount of the current to be injected to the active layer and the intensity of the light output. The amount of change of the light intensity with respect to the amount of the current to be injected to the active layer is larger when the laser oscillates in the multimode than in the single-mode. Therefore, the light intensity is measured at the center position while the amount of the current to be injected to the active layer is gradually increased. From the measured result, the light intensity is determined when a gradient of the amount of change of the light intensity with respect to the amount of the current to be injected to the active layer becomes large. The light intensity in such a case is represented as I_{A}. That is, I_{A} can be determined from a second order differential value of the light intensity with respect to the amount of the current to be injected. This is one of the methods to determine I_{A}. Note that when the gradient of the amount of change of the light intensity with respect to the amount of the current to be injected to the active layer becomes large, there is a possibility that a value of the amount of the current to be injected (current value at the time of a mode change) deteriorates due to time degradation of the active layer. Therefore, it is preferable to set I_{A} lower than a value determined by the above-mentioned method considering the deterioration of the current value at the time of the mode change, which is possible due to the time degradation of the active layer.

A method for estimating the light-intensity distribution of a laser beam with reference to the light intensity I₂ detected at Xᵢ or Xⱼ will be described. For example, by preparing a surface emitting laser, except for the pair of the light receiving portions 150 shown in FIGS. 1A and 1B, the light intensity at each position in the x-direction is measured by a photodetector provided separately from the surface emitting laser. Then, by changing the amount of the current to be injected to the active layer, the light-intensity distribution depending on the amount of the current to be injected can be known by measuring in a similar way. In the surface emitting laser shown in FIGS. 1A and 1B, as long as information of this light-intensity distribution is stored as a data table, the light-intensity distribution can be measured by detecting the optical signals with the pair of light receiving portions 150 and by fitting the detected results to the data table. Note that in a case of measurement with the separately provided photodetector outside the cavity, the light-intensity distribution to be measured is a far field pattern (FFP). Therefore, data regarding correspondence between the light-intensity distribution of the FFP and that of a near field pattern (NFP) is obtained in advance. Then, information of the light-intensity distribution of the NFP depending on the amount of the current to be injected is stored as a data table. Alternatively, the light-intensity distribution of the NFP depending on the amount of the current to be injected is obtained by using a photodetector, which is separately provided outside the cavity (for example, charge-coupled device (CCD)) with an optical system such as a microscope. Then, the information of the light-intensity distribution of the NFP is stored as a data table.

As shown in FIG. 2B, the intensity of the laser beam generally deteriorates due to a certain factor. Herein, the light intensity at X_{c} deteriorates from I₁ to I₁′ and the light intensity at Xᵢ and Xⱼ also deteriorate from I₂ to I₂′. In such a case, the light-intensity distribution from the light intensity I₂′, which is a result detected at Xᵢ or Xⱼ, to the light intensity I₁′, which is a result detected at X_{c} is determined based on the data table. Further, it is determined whether the transverse mode state is the single-mode or the multimode.

As shown in FIG. 2C, light intensity I₂″ of laser beams detected at Xᵢ and Xⱼ are both larger than I_{A}. In such a case, the transverse mode is determined as the multimode with reference to the data table. Further, the light-intensity distribution from the light intensity I₂″ detected at Xᵢ and Xⱼ to light intensity I₁″ at X_{c} can also be determined with reference to the data table. As shown in FIG. 2D, there is a case where the peak position of the light-intensity distribution of the laser beam shifts due to a certain factor.

In such a case, the light intensity I₂′″ detected at Xⱼ is larger than I_{A}, while light intensity detected at Xᵢ is smaller than I_{A}. Accordingly, in a case where the light intensity at either Xᵢ or Xⱼ is larger than I_{A}, the transverse mode is determined as the single-mode instead of the multimode. The light intensity I₁′″ at the center X_{c} and/or a value at the peak position can also be determined from the data table.

In addition, such light-intensity distribution shown in FIG. 2D is likely to appear when an optical path changes. Therefore, it is possible not only to determine the transverse mode state and to estimate the light intensity at the center position but also to distinguish between a case where the transverse mode state is the single-mode and the optical path has changed and a case where the transverse mode state is the multimode. The change of the optical path may possibly occur, for example, when a position of a light-emitting region of the active layer changes due to a change in temperature of the active layer.

In order to maintain a state of the single-mode, a method for controlling the amount of the current to be injected to the active layer can be taken into consideration without detecting the optical signals as described in the present invention. However, the value of the amount of the current to be injected (or the current value at the time of the mode change) which can maintain the state of the single-mode is variable depending on a temperature of a VCSEL and a wavelength of light to be emitted. Therefore, it is difficult to maintain an operating state of the single-mode without detecting the optical signals.

In the embodiment described hereinbefore, it is determined whether the transverse mode is the single-mode or the multimode depending on a magnitude relation of the predetermined value I_{A}. Such determination has been carried out from the optical signals detected by the plurality of light receiving portions located at positions other than the center X_{c}. However, whether the transverse mode is the single-mode or the multimode can be also determined from the optical signals detected by the light receiving portion located in the center X_{c} and from the optical signals detected by the light receiving portions located in positions other than the center. In such a case, a ratio between the light intensity detected at the center X_{c} and the light intensity detected at, for example, Xⱼ other than the center is determined. Then, the ratio is compared with a ratio measured in advance to determine the transverse mode.

On the other hand, in cases where there is one light receiving portion, the intensity of the light output and the operating state of the transverse mode cannot be detected simultaneously. The reason therefor will be described with reference to an example of a case where one light receiving portion is disposed in the above-mentioned Xⱼ. In such a case, the light intensity I₂″ detected at Xⱼ shown in FIG. 2C and the light intensity I₂′″ detected at Xⱼ shown in FIG. 2D are both larger than I_{A}. Therefore, when there is one light receiving portion, it is difficult to determine whether the transverse mode state is the single-mode or the multimode.

Another simpler embodiment for controlling the current to be injected to the surface emitting laser will be specifically described.

In a fixed-wavelength or a variable wavelength surface emitting laser according to the present embodiment, when the current to be injected to the surface emitting laser is changed, the output power of emission (the light intensity) is detected by an external photodetector (such as a large-size photodiode, and an integrating sphere) which is prepared. At the same time, the light intensity at Xᵢ is detected with the light receiving portion located in Xᵢ of this surface emitting laser. Then, a relation between these detected two light intensities is prepared as a table when the current to be injected to this surface emitting laser is changed (in a practical use, the table is stored in a storage unit as hereinafter described). However, there is no separately prepared external photodetector in practically using this surface emitting laser (for example, in using the laser in an optical coherence tomography). Therefore, it is possible to estimate an output power of emission depending on injected current from the surface emitting laser according to the present embodiment, using by the signal of the light receiving portion at Xᵢ and the prepared table.

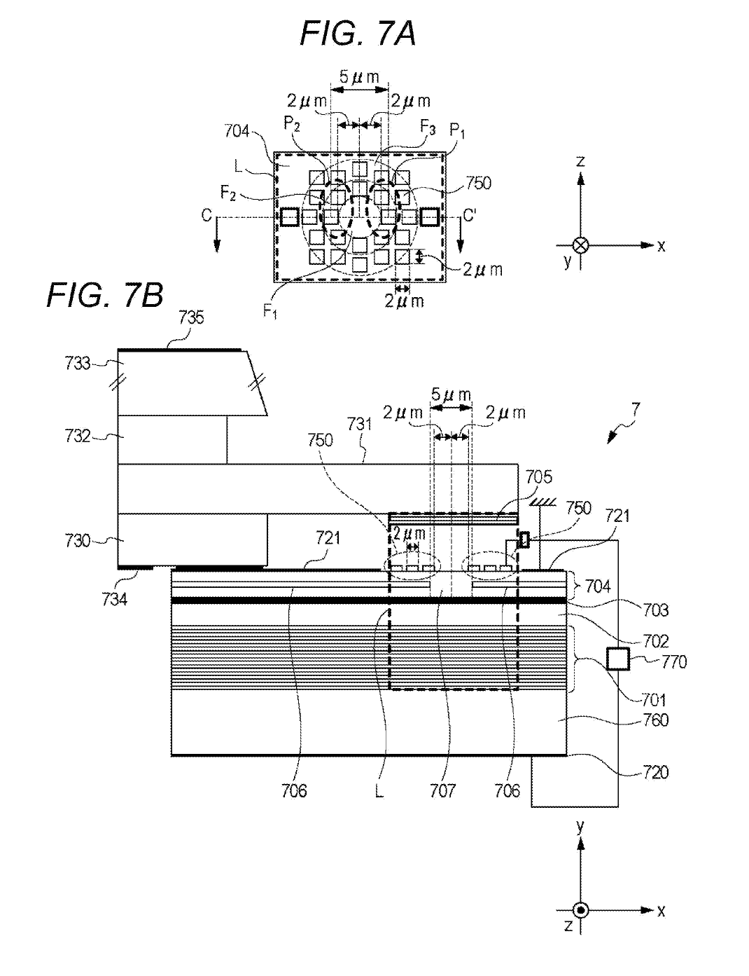

Regarding detection of a mode state, it can be determined whether the mode status is changing depending on a change of signals transmitted from two or more light receiving portions located at positions are equivalent to Xᵢ in FIG. 1A, as a structure shown in FIG. 7A. For example, in cases where the mode changes from the light-intensity distribution of the NFP shown in FIG. 2A to the light-intensity distribution of the NFP shown in FIG. 2C by changing the current to be injected (changes from a single-mode operation to a multimode operation), the signals at Xᵢ and Xⱼ intensify. In such a case where the signals at both Xᵢ and Xⱼ (located at positions equivalent to Xᵢ) intensify, the transverse mode state can be determined as being in the multimode operation. However, the transverse mode can be determined the single mode operation when there is one in all of photodetector shown in FIG. 7A has the signal that intensify when the current to be injected to the surface emitting laser is increased. In such a case, the transverse mode is in the single-mode operation in which the light-intensity distribution of the NFP shown in FIG. 2D, that is, a light-emitting point (peak of light emitting intensity) has shifted from the center of a current confinement structure.

Note that the signal intensity of Xᵢ in the single-mode operation is smaller than the signal intensity of Xᵢ when the kink appears in a characteristic curve of current-output power of emission. This characteristic curve is gotten by external photodetector while the above-mentioned table is prepared. The kink herein is the value of current when inflection point is appeared in the characteristic curve of s second order differential of ΔL/ΔI vs. Current. In FIG. 2A, the signal intensity of Xᵢ is represented as I_{A} when this kink appears.

In a case of the light-intensity distribution of the NFP as shown in FIG. 2B, there is no light receiving portion in which signals stronger than I_{A} are detected. However, the signals from the photodetector disposed at the either position Xᵢ or Xⱼ is smaller than the case shown in FIG. 2A. Therefore, the transverse mode is determined as being in the single-mode operation in which the light output has been deteriorated.

As described above, when there is one or no point of intensity stronger than I_{A}, such a case represents that the transverse mode state is in the single-mode operation. When there are two or more points thereof, it can be determined that the transverse mode state is in the multimode operation. However, it should be noted that when the peak of the light emitting intensity is shifted from the center of the current confinement structure and when the transverse mode state is in the single-mode operation, a plurality of points of the intensity stronger than I_{A} may be measured. However, in such a case, as long as the intensity of a photodetector around a photodetector with the strongest intensity is equal, the transverse mode state can be determined as being in the single-mode operation.

The intensity of light which enters the light receiving portions 150 is measured by the control unit 170 as hereinafter described. The control unit 170 controls current to be injected between the electrodes 121 and 120 based on the optical signals detected by the light receiving portions 150. The state of the transverse mode can also be changed by changing current to be injected to the active layer. For example, in a case of the multimode, the control unit 170 reduces the current to be injected between the electrodes 121 and 120 so as to control the transverse mode state to be the single-mode based on the detected result of the optical signals detected by the light receiving portions 150. In cases where the light intensity at a desired position is determined, by the detected optical signals, as not reaching the desired value, the light output can be increased by enlarging current to be injected between the electrodes 121 and 120. Of course, when the transverse mode state is determined as the single-mode and when it is necessary to lower the light intensity at the desired position, it is sufficient to reduce the current to be injected.

In such a way, the control unit 170 can control the intensity of the light output power of the surface emitting laser and control the transverse mode state to be in a desired state based on photoelectric conversion signals from the plurality of light receiving portions 150.

As described above, in the surface emitting laser according to the present embodiment, accuracy of determining the transverse mode state becomes high. What is more, measurement of the intensity of the light output power and determination of the operating state of the transverse mode can be simultaneously carried out. Further, in the surface emitting laser, the intensity of the light output power can be controlled as a value of a desired range, and the transverse mode state can be controlled to be in the desired state.

As described above, the surface emitting type semiconductor laser disclosed in Patent Literature 1 includes one light absorbing layer (light receiving portion). Therefore, it is difficult to distinguish, with satisfactory accuracy, whether a transverse mode state of light to be emitted is a single-mode or a multimode. That is, the present inventors have found a problem that it is difficult to distinguish between a case where the transverse mode state of the light to be emitted is the single-mode and an optical path has changed, and a case where the optical path is invariable and the transverse mode state has changed from the single-mode to the multimode. Particularly, the above-mentioned problem is likely to occur in a variable wavelength VCSEL, because there is a possibility that an optical path of light in the single-mode changes when an upper reflecting mirror moves.

An object of the present invention is to provide a surface emitting laser which can detect the transverse mode state of the light to be emitted with better accuracy than the related art, and an optical coherence tomography.

### Second Embodiment

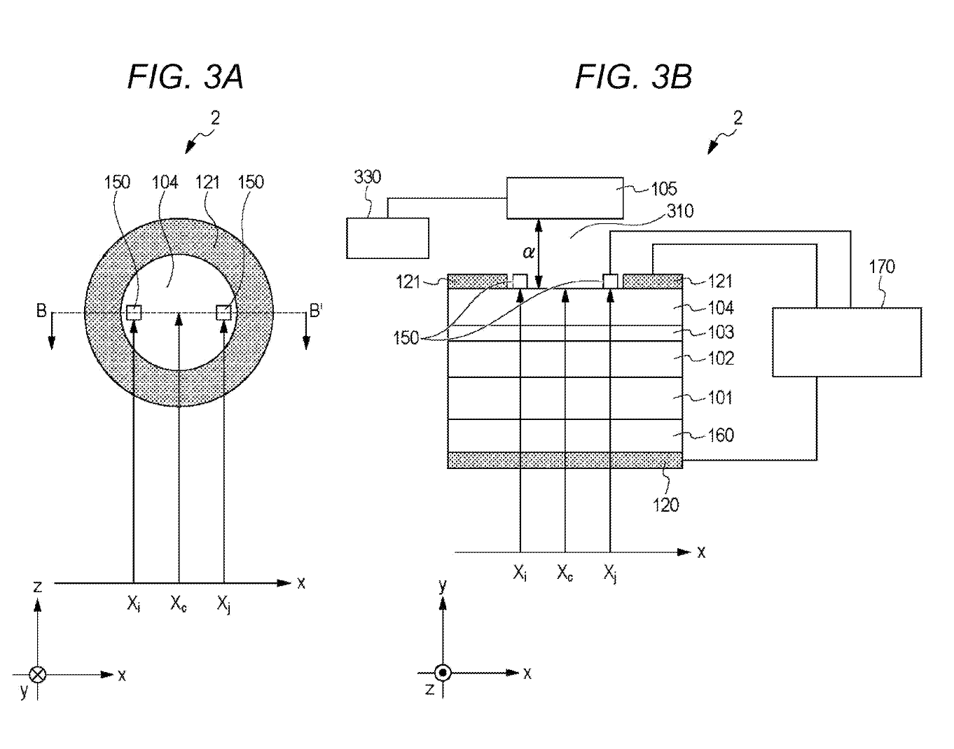

A surface emitting laser according to a second embodiment will be described with reference to FIGS. 3A and 3B. FIG. 3A is a top view of the surface emitting laser according to the present embodiment. FIG. 3B is a cross-sectional view taken along line B-B′ in FIG. 3A. In FIGS. 3A and 3B, the same members as those in FIGS. 1A and 1B will be denoted with the same signs and detailed description thereof will be omitted.

A surface emitting laser 2 according to the present embodiment is a variable wavelength VCSEL. Herein, a second cladding layer 104 and an upper reflecting mirror 105 are separate from each other, and a gap 310 is provided in an optical path. In a case of changing a distance from an interface between the second cladding layer 104 and the gap 310 to an interface between the upper reflecting mirror 105 and the gap 310 (a distance α in FIG. 3B), a cavity length changes. As a result, a wavelength of a laser beam to be oscillated can be changed. Accordingly, in a case of using a controlling mechanism which changes the distance α, it is possible to change the wavelength of the laser beam to be oscillated, that is, an oscillation wavelength in examples shown in FIGS. 3A and 3B.

Moreover, it is possible to sweep the oscillation wavelength at a high speed by making a round trip the upper reflecting mirror 105 at a high speed in the y-direction while the current to be injected to an active layer 103.

Regarding such a variable wavelength VCSEL of the present embodiment, the VCSEL does not always oscillate in the single-mode in which a peak of intensity appears at the center of a light-emitting region. Whether the peak appears at the center depends on a movement of a movable mirror, an error of bonding the movable mirror, variation in producing a light-emitting portion, or an amount of current to be injected in driving the VCSEL. For example, in cases where the movable mirror is driven by a cantilever, the optical path of light which has been making a round trip in the y-direction may change as the cantilever curves. Further, in cases where a region to which current is injected shifts, a position and a dimension of the light-emitting region may change. Therefore, the VCSEL may oscillate in the single-mode in a region shifted from the center of the light-emitting region.

Therefore, even in the variable wavelength type surface emitting laser in the present embodiment, it is preferable to locate the plurality of light receiving portions and to determine the intensity of the light output power and the operating status of the transverse mode. It is possible to achieve a surface emitting laser with stable output by locating the plurality of light receiving portions, and determining the intensity of the light output power as well as the transverse mode state, and controlling the laser to be in a desired state.

Measurement of the intensity of the light output power and determination of the transverse mode state by the light receiving portions are similar to what has been described in the first embodiment. Therefore, description herein will be omitted.

Hereinafter, each constituent element of the fixed-wavelength or variable wavelength surface emitting laser according to the embodiment of the present invention will be described in detail.

### (Light Receiving Portion)

The light receiving portions employed in the present embodiment of the present invention are a plurality of individual light receiving portions for detecting a laser beam. In the present embodiment, Xᵢ and Xⱼ where the plurality of light receiving portions is located are not particularly restricted, as long as the intensity of the light output and the operating state of the transverse mode can be determined therein. As described in the first embodiment, among the circular region encircled by the electrode 121 on the cladding layer 104, it is preferable to locate the light receiving portions 150 in the vicinity of the center X_{c} instead of locating at the center X_{c} where light emitting intensity becomes high. This is because such a case can reduce a loss of the intensity of the light output power.

Further, in cases where the light receiving portion is located at a position where a peak of the light intensity appears, the light at the position where the light receiving portion is located may be absorbed. This results in deterioration of the light intensity at the position, but the light intensity in vicinal positions may be maintained. Therefore, the light-intensity distribution to be output may be deteriorate compared with the original distribution. Accordingly, it is preferable to locate the light receiving portion at a position other than one where the peak of the light intensity appears. However, when the position is too far from X_{c}, it is difficult to detect the change of the transverse mode state. Therefore, it is preferable to locate the light receiving portion at a position where deterioration of the intensity of the light output power to be eventually emitted is reduced and the transverse mode state can be determined with sufficient accuracy.

The positions Xᵢ and Xⱼ in which the light receiving portions are located may be, for example, positions having the light intensity of 1/10 or less of the light intensity detected at X_{c}. More preferably, the positions may have the light intensity of 1/100 or less. The positions with the light intensity of 1/10 or less, or 1/100 or less of the peak value of the light intensity can be located by the following processes as described above. For example, the position can be located by producing a surface emitting laser having the same structure except for not having the light receiving portion, and by measuring the distribution of the light output power with the produced surface emitting laser.

In the embodiment of the present invention, any one of the plurality of light receiving portions 150 may be located on the second cladding layer 104 or on the upper reflecting mirror 105. Alternatively, the plurality of light receiving portions may be located on both the upper reflecting mirror 105 and the second cladding layer 104. In a case where the light receiving portions 150 are located on the second cladding layer 104, the portions may be embedded into the upper reflecting mirror 105 as shown in FIGS. 1A and 1B. Alternatively, the portions may also be located on the second cladding layer 104 while being separated from the upper reflecting mirror 105 as shown in FIGS. 3A and 3B.

Further, it is preferable to locate at least one of the plurality of light receiving portions 150 inside a region having a radius of 10 μm from the center of the optical path over the cladding layer 104. The reason is that the peak of the light intensity is likely to appear from a region having a radius of 10 μm or less from the center of a light emitting position when the operating state changes from the single-mode to the multimode.

Still further, it is preferable to locate at least one of the plurality of light receiving portions 150 outside a region having a radius of 2 μm from the center of the optical path on the cladding layer 104.

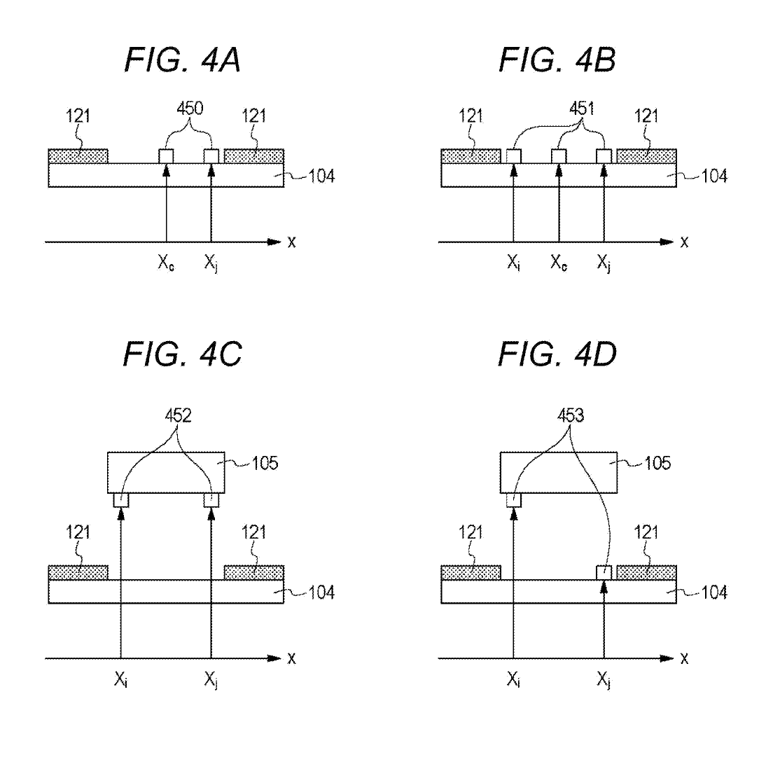

Regarding the surface emitting laser according to the embodiment of the present invention, other examples in which the light receiving portions are located at different positions from those shown in FIGS. 3A and 3B will be described with reference to FIGS. 4A to 4D. Each view of FIGS. 4A to 4D is a modification of a partial structure of the surface emitting laser shown in each of FIGS. 3A and 3B. The same members herein as in FIGS. 3A and 3B will be denoted with the same signs and detailed description thereof will be omitted.

FIG. 4A is a view showing an example in which light receiving portions 450 are located at positions X_{c} and Xⱼ. A method for measuring the intensity of the light output power and determining the operating state of the transverse mode in a structure shown in FIG. 4A will be described with reference to FIGS. 2A to 2D.

In a case of FIG. 2A, when the light intensity detected by the light receiving portion 450 located at Xⱼ is smaller than I_{A}, the transverse mode state is determined as the single-mode.

In a case of FIG. 2C, the transverse mode state is determined as the multimode since the light intensity of a laser beam detected at Xⱼ is larger than I_{A}.

In a case of FIG. 2D, the light intensity I₂′″ detected at Xⱼ is larger than I_{A}. In this case, the transverse mode state is determined as the single-mode, not the multimode, and as an oscillation state in which the optical path has changed.

In this manner, even in the structure shown in FIG. 4A, the intensity of the light output power can be measured and the transverse mode state can be determined at the same time.

In cases where the light-intensity distribution in FIG. 2C shifts in a forward direction of an x-axis and where the light intensity of I₂″ and I₁″ are respectively detected at X_{c} and Xⱼ, the light receiving portions may be located in the forward direction of the x-axis further than Xⱼ. Further, the transverse mode state may be determined as the multimode and the optical path may be determined as being changed.

FIG. 4B is a view showing an embodiment in which three light receiving portions 451 are respectively located at X_{c}, Xᵢ and Xⱼ. Compared to the above-mentioned case with two light receiving portions, the light-intensity distribution can be measured with better accuracy in this embodiment. Further, a data table herein can be made simpler than the case described above. In a case of this embodiment, objects to be determined may be separated by using photoelectric conversion signals from the light receiving portions. For example, the light intensity may be detected by the light receiving portion located at X_{c} to measure the intensity of the light output power from the detected light intensity, and the transverse mode may be determined by the light receiving portions located at Xᵢ and Xⱼ.

Further, it is not necessary to locate the plurality of light receiving portions in a direction vertical to a surface of a substrate. More specifically, as shown in FIG. 4C, light receiving portions 452 may be located at positions Xᵢ and Xⱼ on an upper reflecting mirror 105. Alternatively, as shown in FIG. 4D, the surface emitting laser may be configured to include light receiving portions 453 located at position Xᵢ on the upper reflecting mirror 105 and at position Xⱼ on the second cladding layer 104.

Further, the number of the plurality of light receiving portions may be three or more, and those portions may be arranged on surfaces which intersect with an irradiation direction of a laser beam. That is, three light receiving portions may be arranged at two-dimensional positions so as not to be arranged linearly. Details of such an example will be hereinafter described.

A structure of the light receiving portions according to the present embodiment is not particularly restricted as long as the light intensity can be detected therein. As a light receiving portion which operates as a photodetector, a photoelectric conversion element such as a photovoltaic element can be used. Further, the photoelectric conversion element may be produced separately from the surface emitting laser and may be attached to the surface emitting laser.

Alternatively, the photoelectric conversion element may be configured to form layers other than those included in the surface emitting laser. As those layers cooperate, the photoelectric conversion element herein may carry out a photoelectric conversion operation. That is, in the photoelectric conversion element, a first cladding layer and a second cladding layer may consist of semiconductor layers. Further, at least one of a plurality of light receiving portions herein may be adjacent to the first cladding layer or the second cladding layer and may consist of a semiconductor layer with a conductivity type different from that of the adjacent first cladding layer or the second cladding layer. For example, the second cladding layer may be a first conductivity type (p-type or n-type) semiconductor layer, and a layer having a second conductivity type (n-type or p-type) semiconductor layer different from the first conductivity type may be located on the second cladding layer. In such an embodiment, a p-n junction portion consisting of the first conductivity type second cladding layer and the second conductivity type semiconductor layer located on the second cladding layer functions as a photodiode.

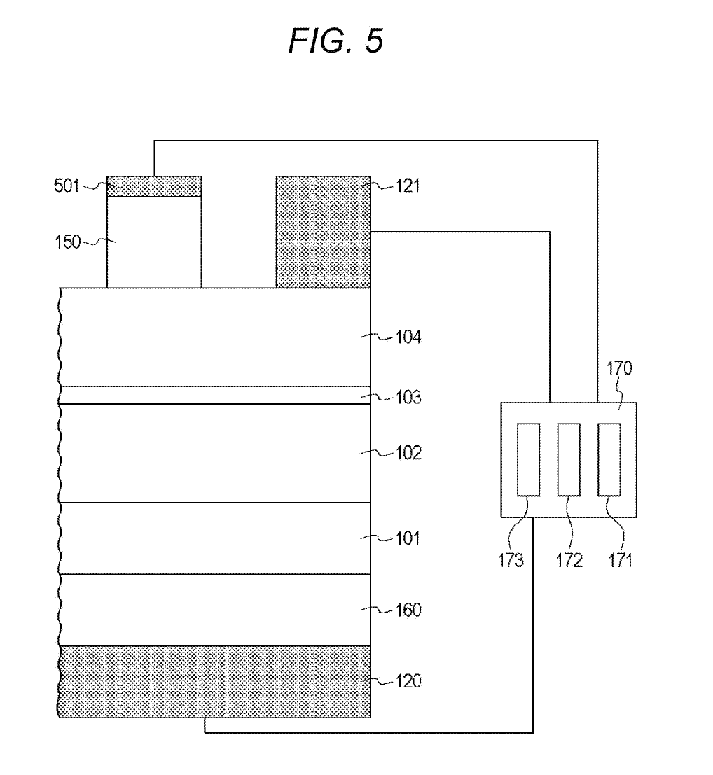

An exemplary method for detecting optical signals and driving a laser according to the embodiment of the present invention will be described with reference to FIG. 5. The same members as those in FIGS. 3A and 3B will be denoted with the same signs in FIG. 5 and detailed description thereof will be omitted.

FIG. 5 is an enlarged view of a part of the surface emitting laser in FIG. 1B. Herein, a second cladding layer 104 consists of a p-type semiconductor, while a light receiving portion 150 consists of an n-type semiconductor in order to provide a p-n junction photodiode. An electrode 501 and an electrode 121 are connected to a control unit 170.

The control unit 170 consists of a measurement unit 171, a storage unit 172, and a difference detecting unit 173. Materials constituted the light receiving portion 150 may be one having a band gap which is capable of absorbing a laser beam. For example, a compound semiconductor such as GaAs and InGaAs may be used as the materials. A composition ratio of each layer of the light receiving portion can be appropriately selected depending on a wavelength band of light to be detected. In cases where the n-type semiconductor is used as the second cladding layer 104, the p-type semiconductor may be used as the light receiving portion 150.

First, light which has passed through the second cladding layer 104 enters the light receiving portion 150 which has a band gap narrower than that of the second cladding layer 104. Such optical absorption results in generating a pair of electron-hole pair. The electron passes through the n-type semiconductor light receiving portion 150 and reaches the electrode 501 located on the light receiving portion 150. On the other hand, the hole passes through the p-type semiconductor second cladding layer 104 and reaches the electrode 121. The measurement unit 171 measures photocurrent which flows between the electrode 501 and the electrode 121. Note that positive voltage is applied to the electrode 501, while reverse bias is applied to the light receiving portion 150. The storage unit 172 stores, as a data table, a relation between the photocurrent, as photoelectric conversion signals from the light receiving portion, and the light intensity. The photocurrent measured by the measurement unit 171 can be converted into the light intensity by using the data table. The difference detecting unit 173 detects a difference between the converted light intensity and a predetermined light intensity. Based on the difference, the difference detecting unit 173 changes the current to be injected between the electrode 121 and an electrode 120 included in the surface emitting laser.

For example, as shown in FIG. 2C, by setting the predetermined light intensity as I_{A}, the transverse mode can be determined as the multimode when the detected light intensity becomes I₂. Therefore, current to be injected between the electrode 121 and the electrode 120 is reduced, and the transverse mode is transferred to the single-mode. Then, it is determined whether the light intensity at the center is within a predetermined range, or it is determined whether the intensity of the light output power is within the predetermined range. Then, a driving current may be adjusted so as to be changed further.

In such a way, measuring the intensity of the light output power and determining the transverse mode of the surface emitting laser according to the present embodiment can be carried out at the same time based on the measured light intensity.

The example in which the photocurrents are measured as the photoelectric conversion signals has been described. However, the measurement unit 171 may be one which measures photovoltaic power from the light receiving portion. Note that the control unit 170 may preferably change driving current of the surface emitting laser, but may change the driving voltage.

### (Upper Reflecting Mirror and Lower Reflecting Mirror)

In the present embodiment, the cavity is composed of an upper reflecting mirror and a lower reflecting mirror. Those mirrors are not particularly restricted as long as each mirror has a necessary reflection ratio. For example, a multilayer mirror may be used as well. In the multilayer mirror, a low refractive index layer and a high refractive index layer are alternately laminated. Note that at least the upper reflecting mirror has a reflection ratio at which a laser beam can pass through the mirror.

Further, high contrast grating (HCG) mirrors can be used as the upper reflecting mirror and the lower reflecting mirror according to the present embodiment. The HCG mirror is composed of a high refractive index material and a low refractive index material. The materials herein are alternatively and periodically located in an in-plane direction. Examples of the HCG mirror include a periodic structure that consists of a high refractive index region (AlGaAs) and a low refractive index region (slits). Herein, a semiconductor layer such as an AlGaAs layer is processed to provide periodic slits. It should be noted that structures and materials of each upper reflecting mirror and lower reflecting mirror according to the present embodiment can be selected individually.

In a case of the variable wavelength VCSEL, it is preferable to use a light mirror as a moved reflecting mirror (the upper reflecting mirror in FIG. 1B) to sweep the wavelength fast. Therefore, as the upper reflecting mirror, it is preferable to use the HCG mirror with a thin (light) structure instead of the multilayer mirror with a thick (heavy) structure.

Examples of a dielectric multilayer mirror include a dielectric multilayer consisting of a plurality of pairs of SiO₂ layers as silicon oxide layers and TiO₂ layers as titanium oxide layers. On the other hand, examples of a semiconductor multilayer mirror include a semiconductor multilayer consisting of a plurality of pairs of AlGaAs layers and GaAs layers. Note that a bandwidth of a high-reflection ratio and a reflection ratio thereof can be controlled by appropriately changing the number of pairs of the multilayer mirrors.

Further, in the embodiment according to the present invention, a structure of the movable mirror may be a micro electro mechanical system (MEMS) structure used in a silicon cantilever which drives using an electrostatic attraction, and in the HCG mirror having a beam structure.

### (Active Layer)

An active layer according to the present embodiment is not particularly restricted as long as a material thereof emits light through current injection. In cases where light with a wavelength band of nearly 850 nm is emitted, a material having a quantum well structure including AlₙGa₍₁₋ₙ₎As (n represents a number of 0 or more and 1 or less) can be used. Further, in cases where light with a wavelength band of nearly 1050 nm is emitted, a material having a quantum well structure including InₙGa₍₁₋ₙ₎As (n represents a number of 0 or more and 1 or less) can be used. Further, the active layer may be composed of a single quantum well or a multi quantum well.

### (First Cladding Layer and Second Cladding Layer)

In the embodiment of the present invention, cladding layers are located in order to confine light and carrier. Further, in the embodiment of the present invention, the cladding layers also assume a role as a spacer in order to adjust the cavity length.

Examples of the first cladding layer and the second cladding layer according to the present embodiment may consist of a lamination layer of an AlGaAs layer in which a composition of Al has been appropriately selected depending on a wavelength band of the light to be emitted, and an AlGaAs layer in which the composition of Al has been changed. For example, in cases where light with a wavelength band of nearly 850 nm is emitted, an Al_{0.8}GaAs layer may be used. In cases where light with a wavelength band of nearly 1050 nm is emitted, a lamination layer of an Al_{0.4}GaAs layer and a GaAs layer may be used. Note that the first cladding layer and the second cladding layer are different in a conductivity type. Regarding the cavity length, in the fixed-wavelength VCSEL, the thickness of the cladding layer is adjusted in order to ensure a sufficient cavity length because the fixed-wavelength VCSEL may consist of a λ cavity or a long cavity of about 5λ. On the other hand, in the variable wavelength VCSEL, it is preferable to include a 3 to 4λ cavity considering a movable region (or gap hereinafter described) of the movable mirror, drive thereof, or a current confinement structure. In order to obtain such a preferable cavity, the thickness of the cladding layer herein is adjusted. Note that the thickness of the first cladding layer may not necessarily be the same as that of the second cladding layer when the thicknesses of the cladding layers are adjusted. The thickness can be appropriately selected as long as the cavity length can be adjusted.

### (Current Blocking Layer)

In the embodiment of the present invention, a current confinement layer can be formed as required in order to restrict a region where current injected to the laser flows. The current confinement layer is formed by implantation of a hydrogen ion, or by selective oxidization of an AlGaAs layer located inside the cladding layer and having the Al composition of 90% or more. It is preferable to locate at least one of the plurality of light receiving portions on the current confinement layer. It is further preferable to locate the plurality of light receiving portions at positions which do not overlap one electrode (for example, the above-mentioned electrode 121 and an electrode 721 hereinafter described) in the x-direction. As a result, density of the current to be injected can be heightened by not involving the current confinement layer. Then, a position at which the peak of the light intensity appears, and positions at which the light receiving portions are located can be shifted from each other.

### (Control Unit)

In the embodiment of the present invention, as described above, a control unit consists of a driving circuit and a measuring circuit. The driving circuit is for driving the surface emitting laser 1. The measuring circuit is for measuring the intensity of a laser beam by individually receiving photoelectric conversion signals from the plurality of light receiving portions. The measuring circuit herein is a circuit for detecting the transverse mode of the laser beam and the plurality of light receiving portions is connected thereto.

Further, a feedback loop is formed in order to feed back the signals detected by the plurality of light receiving portions to drive the surface emitting laser. In such a way, the driving current of the surface emitting laser can be changed based on the plurality of photoelectric conversion signals detected by the plurality of light receiving portions.

Further, in order to determine whether the transverse mode state of the laser beam is the single-mode or the multimode, at least two types of the detected photoelectric conversion signals are fitted to measure the light-intensity distribution and the intensity (integrated value) of the light output power of the laser beam. When such functions can be put into practice, the control unit does not need to be hardware consisting of a measurement unit, a storage unit, and a difference detecting unit. Such functions can be achieved through software based on a program which is executed by a microprocessor.

### (Gap)

Regarding a gap according to the present embodiment, a solid usually does not exist. Accordingly, the atmosphere of gap may be vacuum, air, insert gas, or liquid such as water. Note that a gap length (in the y-direction) can be determined considering a variable wavelength bandwidth and pull-in of the movable mirror. For example, the gap length is about 1 μm in the 3 to 4λ cavity including an airy gap. Herein, the 3 to 4λ cavity varies within a variable wavelength bandwidth of 100 nm while centering on 1050 nm.

### (Optical Coherence Tomography)

An optical coherence tomography (hereinafter abbreviated to “OCT”) employing a variable wavelength optical source does not use a spectroscope. Therefore, it is expected to obtain a tomographic image with low loss of light amount and a high S/N ratio.

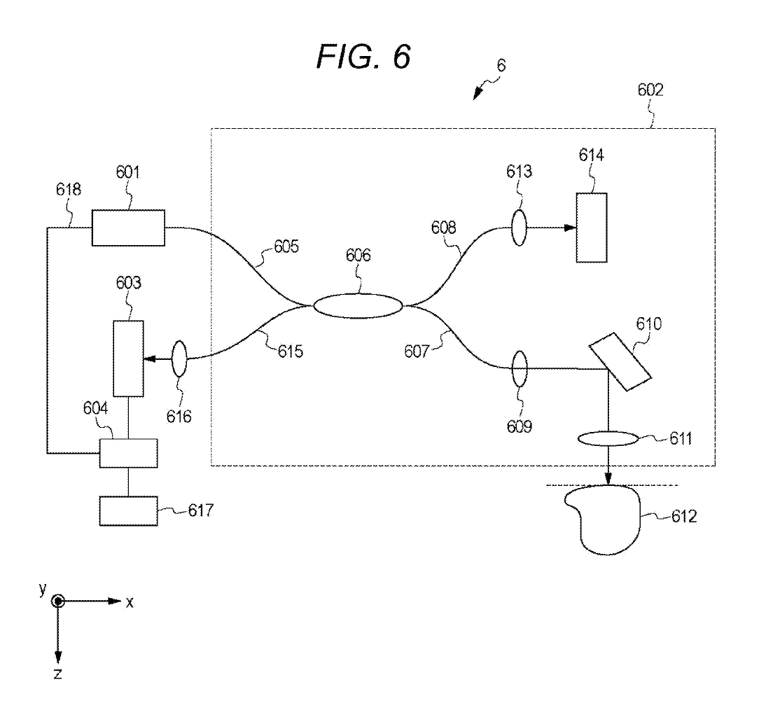

An example in which the surface emitting laser according to the embodiment is used in an optical source of the OCT will be described with reference to FIG. 6.

An OCT apparatus 6 according to the present embodiment comprises at least an optical source 601, an optical coherence system 602, a light detecting unit 603, and an information acquisition unit 604. The above-mentioned surface emitting laser can be used as the optical source 601. Although not shown in the drawing, the information acquisition unit 604 has a Fourier transformer. Although the information acquisition unit 604 herein has been described to have the Fourier transformer, an embodiment is not restricted thereto as long as the information acquisition unit 604 has a function of carrying out Fourier transformation to input data. An example of such a case is where the information acquisition unit 604 has a computation unit, and the computation unit has the function of Fourier transformation. More specifically, the example of such a case is where the computation unit is a computer having a CPU, and the computer executes an application having the function of Fourier transformation. Another example is a case where the information acquisition unit 604 has a Fourier transform circuit having the function of Fourier transformation. Light emitted from the optical source 601 passes through the optical coherence system 602 and is output as coherent light having information of an object 612 to be measured. The coherent light is received by the light detecting unit 603. Note that the light detecting unit 603 may be of a differential detector type or may be of a simple intensity monitor type. Information about time waveform of intensity of the received coherent light is transmitted to the information acquisition unit 604 from the light detecting unit 603. The information acquisition unit 604 obtains a peak value of the time waveform of the intensity of the received coherent light and carries out Fourier transformation. Then, information (for example, tomographic information) of the object 612 is obtained. Note that the above-mentioned optical source 601, the optical coherence system 602, the light detecting unit 603, and the information acquisition unit 604 can be located arbitrarily.

Hereinafter, processes in which light oscillates from the optical source 601 and in which the tomographic information of the object to be measured is obtained will be described in detail.

The optical source 601 changes a wavelength of light. Light emitted from the optical source 601 enters a coupler 606 through a fiber 605 and branches into irradiation light and reference light. The irradiation light passes through a fiber 607 for irradiation light, while the reference light passes through a fiber 608 for reference light. The coupler 606 consists of a coupler which operates in the single-mode in a wavelength band of an optical source. Each type of the fiber coupler may consist of a 3 dB coupler. The irradiation light passes through a collimator 609 to become collimating light, and is reflected by a mirror 610. The light reflected by the mirror 610 passes through a lens 611. Then, the object 612 is irradiated with the reflected light. The reflected light is reflected by each layer in a depth direction of the object 612. On the other hand, the reference light passes through a collimator 613 and is reflected by a mirror 614. In the coupler 606, coherent light occurs due to the reflected light from the object 612 and the reflected light from the mirror 614. The coherent light passes through a fiber 615 and is collected through a collimator 616 and is received by the light detecting unit 603. Information of the intensity of the coherent light received by the light detecting unit 603 is converted into electric information such as voltage and is transmitted to the information acquisition unit 604. In the information acquisition unit 604, data of the intensity of the coherent light is processed. More specifically, Fourier transformation is carried out to obtain tomographic information. Usually, the data of the intensity of the coherent light to which Fourier transformation is carried out are data sampled with an interval of the same wavenumber. However, the data may be sampled with an interval of the same wavelength.

The obtained tomographic information may be transmitted to an image display unit 617 from the information acquisition unit 604 and may be displayed as an image. Note that a three dimensional tomographic image of the object 612 to be measured can be obtained by scanning the mirror 610 inside a plain surface vertical to a direction in which the irradiation light is incident. Further, the information acquisition unit 604 may control the optical source 601 through an electric circuit 618. Although it is not shown in the drawing, the intensity of light emitted from the optical source 601 may be monitored sequentially, and the data may be used for correcting amplitude of signals of the intensity of the coherent light.

The surface emitting laser according to the embodiment of the present invention can measure the intensity of the light output and limit the intensity within the desired range. Therefore, in cases where the laser is used in the OCT apparatus, a tomographic image having a sufficient S/N ratio can be obtained.

Further, the surface emitting laser according to the embodiment of the present invention can obtain a stable output because the transverse mode of light to be emitted can be maintained at the single-mode by following the above-mentioned method.

The OCT apparatus (OCT system) according to the present embodiment is useful for obtaining information on a tomographic image of an organism such as an animal and a human in fields such as ophthalmology, dentistry and dermatology. The information on the tomographic image of the organism includes not only the tomographic image of the organism but also numeric data necessary for obtaining a tomographic image.

Specifically, it is preferable to use the OCT apparatus herein for obtaining information on a tomographic image of the fundus of a human eye as an object to be measured.

### (Other Usage)

The surface emitting laser according to the embodiment of the present invention can be used as an optical source for optical communication and as an optical source for optical measurement other than the above-mentioned OCT.

### EXAMPLES

Hereinafter, Examples of the present invention will be described.

### Example 1

In Example 1, a variable wavelength VCSEL as a variable wavelength surface emitting element will be described. The VCSEL herein has a movable mirror with a plurality of light receiving portions inside a cavity. The structure of the VCSEL is a cantilever type MEMS structure.

FIGS. 7A and 7B are schematic views for explaining a structure of the variable wavelength VCSEL according to the present Example. FIG. 7A is a top view of the variable wavelength VCSEL according to the present Example, while FIG. 7B is a cross-sectional view taken along line C-C′ in FIG. 7A. FIG. 7A is a view showing a vicinity of the light receiving portions when the variable wavelength VCSEL in FIG. 7B except for the movable mirror is seen from the upper direction (upper direction on a page).

In FIG. 7B, an n-type multilayer mirror 701 is disposed on an n-type semiconductor substrate 760 consisting of a GaAs layer as a group III-V compound semiconductor. In the n-type multilayer mirror 701, 45 pairs of Al_{0.8}GaAs layers (68.1 nm in thickness) and Al_{0.3}GaAs layers (62 nm in thickness) as the group III-V compound semiconductor are laminated.

On the multilayer mirror 701, an n-type cladding layer 702 consisting of an Al_{0.8}GaAs layer (102.6 nm in thickness) is located. On the n-type cladding layer 702, an active layer 703 is located. The active layer 703 has a triple quantum well structure consisting of a combination of a GaAs well layer (10 nm in thickness) and an Al_{0.3}GaAs barrier layer (10 nm in thickness). Further, a p-type cladding layer 704 consisting of an Al_{0.8}GaAs layer (337.4 nm in thickness) is located on the active layer 703.

On the p-type cladding layer 704, a plurality of n-type light receiving portions 750 consisting of a GaAs layer (25 nm in thickness) is located.

Further, this variable wavelength VCSEL 7 has an anode 721 and a cathode 720. The anode is for driving a laser and for measuring photoelectric conversion signals from the light receiving portions 750. The cathode 720 is for driving a laser. Moreover, the variable wavelength VCSEL 7 is connected to a current variable drive power source 770 as a control unit.

A movable mirror 705 is located on the bottom surface of a leading end of a silicon cantilever 731 (2 μm in thickness). Further, the movable mirror 705 is supported by a silicon oxide layer 730 (1 μm in thickness), a silicon cantilever (2 μm in thickness) 731, a silicon oxide film (2.5 μm in thickness) 732, and a silicon substrate 733 on a substrate 760 via several layers between the substrate 760 and the above-mentioned members. The movable mirror 705 is a dielectric multilayer mirror in which ten pairs of SiO₂ layers (145.5 nm in thickness) and TiO₂ layers (90 nm in thickness) are laminated. In FIGS. 7A and 7B, an optical path inside a cavity is represented by a region L (shown with dotted lines) inside the cavity including the upper reflecting mirror 705 and the lower reflecting mirror 701. Note that the thickness of the silicon oxide layer 730 is the thickness of a gap, and a cavity length when the movable mirror is not driven is 3λ. Further, a Ti/Au electrode 734 and a Ti/Au electrode 735 are formed in order to apply voltage to drive the silicon cantilever 731 by electrostatic attractive force.

In the present Example, the movable mirror 705 is located on the bottom surface of the leading end of the silicon cantilever 731. However, the movable mirror 705 may be located on the upper surface thereof and then, a part of the leading end of the silicon cantilever 731 may be removed.

As shown in FIG. 7A, 26 light receiving portions 750 are located on the top surface of the cladding layer 704. Among those 26 light receiving portions, eight of them are inside a region F₂, twelve of them are inside a region F₃, two of them are outside the region F₃, and four of them are located both inside and outside the region F₃. Note that any of the light receiving portions is not located in a region F₁ which includes the center.

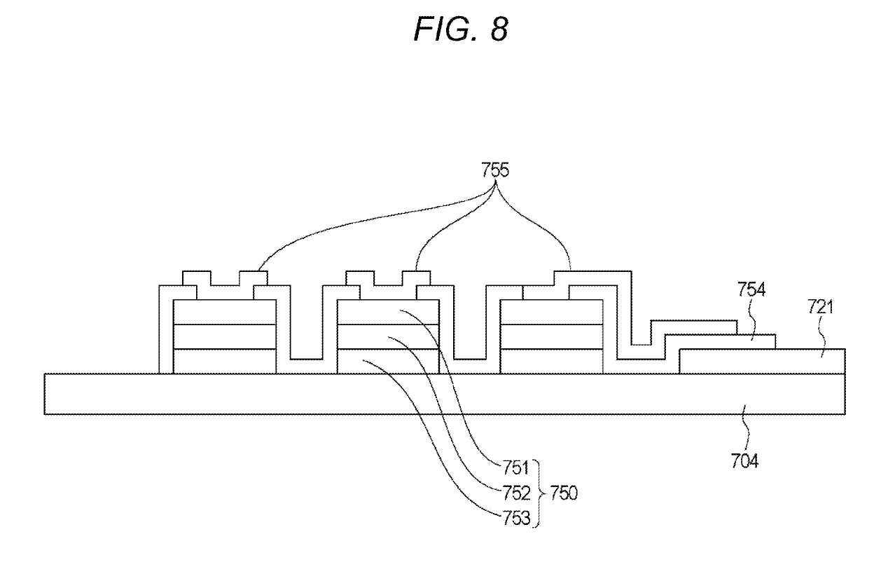

FIG. 8 is a view showing a schematic cross-sectional view of a vicinity of the plurality of light receiving portions 750. Each of the light receiving portions 750 in the present Example is a p-i-n photodiode consisting of a p-layer (p-Al_{0.8}GaAs, 50 nm in thickness) 753, an i-layer (i-GaAs, 50 nm in thickness) 752, and an p-layer (n-Al_{0.3}GaAs, 100 nm in thickness) 751, and is located on the top of the cladding layer 704. Further, each p-i-n photodiode is connected to an individual cathode 755 and the common electrode 721. The individual cathode 755 is for detecting so as to read the photoelectric conversion signals. The common electrode 721 combines an anode of a laser with an anode of photodiode. In FIG. 8, insulation layers 754 (silicon oxide, 200 nm in thickness) are located between each p-i-n photodiode, and elements of the plurality of light receiving portions 750 are mutually isolated. A bias power source applies reverse bias voltage to each p-i-n photodiode. In FIGS. 7A and 7B, the photoelectric conversion signals can be measured by a measurement unit (not shown).

Further, the cladding layer 704 has the current confinement layer 706 which is formed by ion implantation of proton in a part of the p-type cladding layer 704. Therefore, current supplied from the electrode 721 passes through an aperture 707 of the current confinement layer 706 and is injected to the active layer 703. In FIG. 7B, one light receiving portion 750 is wired, but it should be noted that other light receiving portions 750 are also wired. As an electrode for driving the variable wavelength VCSEL of the present Example, the electrode 721 uses a metallic multilayer consisting of a Ti layer (20 nm) and an Au layer (100 nm), while the electrode 720 uses a metallic multilayer consisting of Ni (20 nm), Au (100 nm), and a mixed crystal of Au and Ge (100 nm). Further, a metallic multilayer consisting of a Ti layer (20 nm) and an Au layer (100 nm) is used as electrodes 734 and 735 for driving the movable mirror for sweeping in a wavelength band of ±50 nm centering on a wavelength of 850 nm.

In the present Example, a silicon MEMS structure is used in a moving unit in which a light emitting mirror 705 (upper mirror) is located. The silicon MEMS structure is formed by processing a silicon-on-insulator (SOI) substrate. The variable wavelength VCSEL has the driving unit which is connected with the compound semiconductor substrate 760 on which the lower multilayer mirror (semiconductor DBR mirror) 701, the lower cladding layer 702, the active layer 703, the upper cladding layer 704, and the plurality of light receiving portions 750 are formed.

In the present Example, a light-emitting region determined by a region implanted with proton, that is, the aperture 707 having the current confinement structure provided by ion implantation of proton is formed in a circle shape having a diameter of 5 μm. Any light receiving portion 750 is not located in the circular region F₁ having a radius of 2 μm from the center of the light-emitting region. The plurality of light receiving portions 750 is located in the ring-shaped region F₂ having a radius of 2 to 5 μm, and in the ring-shaped region F₃ having a radius of 5 to 8 μm, both ring-shaped regions located around this circular region. The plurality of light receiving portions 750 is further located in a region outside the regions represented by F₂ and F₃. Each light receiving portion is wired (not shown) so as to be driven individually. By locating the plurality of light receiving portions 750 in such a way, light receiving portions which are capable of observing light-intensity distribution corresponding to the near field pattern (NFP) can be provided. Signal intensities transmitted from the plurality of light receiving portions correspond to the light-intensity distribution corresponding to the NFP. As a result, it is possible to determine the state of the transverse mode (whether it is the single-mode or the multimode).

Hereinafter, a process for measuring the intensity of the light output and for determining the transverse mode of the surface emitting laser according to the present Example will be described with reference to FIGS. 2A and 2B.

First, in a case of the light-intensity distribution shown in FIG. 2A, optical signals corresponding to the light intensity I₂ are detected at a plurality of light receiving portion located in the region F₂. In this case, since I₂ is smaller than I_{A} (I₂<I_{A}), the transverse mode is determined as operating in the single-mode. Further, the intensity of the light output can be calculated from the value of I₂ by fitting with the data table.

Next, in a case of the light-intensity distribution shown in FIG. 2C, optical signals corresponding to the light intensity I₂″ are detected at a light receiving portion group P₁ and at a light receiving portion group P₂ within the region F₂. In this case, since I₂″ is larger than I_{A} (I₂″>I_{A}), the transverse mode is determined as operating in the multimode.

Next in a case of the light-intensity distribution shown in FIG. 2D, it is assumed that, among the light receiving portions within the region F₂, optical signals corresponding to the light intensity I₂′″ are detected at a part of the light receiving portion group P₁, but not at the light receiving portion group P₂. In such a case, it can be assumed that the light-intensity distribution corresponds to the NFP shown in FIG. 2D, and it can be determined that emitted light is the single mode operation. The emitted light herein has a unimodal peak at a point away from the center of the current confinement region. Since the plurality of light receiving portions is located in such a way, the light-intensity distribution corresponding to the NFP away from the center corresponds to signal-intensity distribution from the plurality of light receiving portions. Therefore, compared to a case where a single light receiving portion is located, it is possible to improve accuracy with which to determine a mode status.

Further, in the present Example, a plurality of light receiving portions is located in the region F₃ further outside F₂ and in the region further outside F₃. Therefore, a higher dimensional multimode operation, in which the peak appears in the region F₃, can be detected. Further, by disposing three or more light receiving portions as shown in FIGS. 7A and 7B, each light receiving portion may have a different function from the others. For example, some may be used for monitoring the intensity of the light output power, while the others may be used for monitoring the transverse mode state.

As described hereinbefore, stable driving can be achieved through driving with variable wavelength by locating a plurality of light receiving portions inside the cavity of the variable wavelength VCSEL, monitoring the signal intensity from a plurality of light receiving portions, and adjusting an amount of current to be injected to the light-emitting portion. As a result, the light output within a variable wavelength range can be limited to a range of 5±0.5 mW and all wavelength regions can be operated in the single-mode.

Next, a method for producing the variable wavelength VCSEL of the present Example will be described.

First, the n-type semiconductor multilayer mirror 701, the n-type cladding layer 702, the active layer 703, and the p-type cladding layer 704 are laminated in series on the n-type semiconductor substrate 760 consisting of the GaAs layer by using MOCVD crystal growth technology.

Next, a silicon oxide film is formed on the p-type cladding layer 704. Then, the silicon oxide film is processed by photolithography technology and etching technology so as to function as a mask during proton implantation for forming a current confinement structure. After forming this silicon oxide film mask (not shown), proton implantation is carried out to form the current confinement structure. As another method for forming the current confinement structure, an AlGaAs layer (30 nm in thickness) having an Al composition of 90% or more may be intervened inside the cladding layer 704. The intervened layer may be selectively oxidized from its side surface in an x-direction and may be converted into aluminum oxide to form a high-resistivity region.

Next, after removing the silicon oxide film mask, a p-type Al_{0.8}GaAs layer 753, an i-type GaAs layer 752, and an n-type Al_{0.3}GaAs layer 751 are laminated in series to form the light receiving portion 750. Then, a resist patter for etching is formed on the n-type Al_{0.3}GaAs layer 751. The p-type Al_{0.8}GaA layer 753, the i-type GaAs layer 752, and the n-type Al_{0.3}GaAs layer 751 are etched so as to transfer the resist pattern. Then, 26 island-shaped parts (light receiving portions) each functioning as a p-i-n photodiode are formed. The etching herein may be either dry etching or wet etching.

Next, a metallic layer 721 is formed by photolithography technology, vacuum deposition technology, and liftoff technology. The metallic layer 721 herein serves both as a common electrode and as a connector with the movable mirror (hereinafter simply abbreviated to “common electrode”).

Next, in order to form the individual cathode 755 of the plurality of light receiving portions 750, a surface of the VCSEL in which the common electrode 721 is formed is covered with a silicon oxide film (not shown). After that, a part of the n-type Al_{0.3}GaAs layer 751 of the light receiving portion is exposed by the photolithography technology and the etching technology. Further, a part of the metallic layer 721 which combines the common electrode with the connector with the movable mirror is exposed as well. Then, the individual cathode 755 is formed by the photolithography technology, the vacuum deposition technology, and the liftoff technology.

Next, the cathode 720 for driving the VCSEL is formed on the back surface of the semiconductor substrate 760 by the vacuum deposition technology to achieve a compound semiconductor light-emitting element. Note that positive voltage is applied to the cathode 755 of the light receiving portion, while negative voltage is applied to the cathode 720 for driving the VCSEL.

Alternatively, the conductivity type of each semiconductor layer in the above-mentioned Example may be reversed. That is, the p-type semiconductor layer may be the n-type semiconductor layer, and the n-type semiconductor layer may be the p-type semiconductor layer. As a dopant of the p-type semiconductor layer, Zn can be used. As a dopant of the n-type semiconductor layer, C can be used, but the dopants are not restricted thereto.

The variable wavelength VCSEL of the present Example is assumed to sweep in the wavelength band of ±50 nm centering on a wavelength of 850 nm, but it is not restricted thereto. As hereinafter described in Example 2, the variable wavelength VCSEL may sweep the wavelength in a wavelength band of ±50 nm centering on a wavelength of 1 μm, by appropriately selecting a material of each layer.

### Example 2

In the present Example, a material composition of each semiconductor layer included in the surface emitting laser of Example 1 is changed. Herein, the variable wavelength VCSEL as a surface emitting laser capable of sweeping wavelength in a wavelength band of 1 μm will be described. A basic structure of the variable wavelength VCSEL herein is similar to the structure described in Example 1. Differences in the structure between Examples 1 and 2 will be hereinafter described. In the present Example, more specifically, a DBR consisting of a GaAs layer (71.9 nm in thickness) and an AlAs layer (89.9 nm in thickness) is used as the n-type lower reflecting mirror 701 on the substrate 760. Further, an Al_{0.4}GaAs layer (74.6 nm in thickness) and a GaAs layer (50 nm in thickness) are used as the first n-type cladding layer 702. Further, a multi quantum well layer consisting of an In_{0.32}GaAs well layer (8 nm in thickness) and a GaAs barrier layer (10 nm in thickness) is used as the active layer 703. Moreover, a GaAs layer (50 nm in thickness) and an Al_{0.4}GaAs layer (470 nm in thickness) are used as the p-type second cladding layer 704.

An n-type semiconductor consisting of an InGaAs layer (25 nm in thickness) is used as the light receiving portion 750. As a result of configuring the VCSEL in such a structure, it is possible to sweep a wavelength in a wavelength band of ±50 nm centering on a wavelength of 1 μm.

According to the surface emitting laser of an embodiment of the present invention, the plurality of light receiving portions each capable of individually detecting a laser beam is located in the optical path within the cavity. Therefore, the transverse mode of the laser beam can be detected with better accuracy.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-153784, filed Jul. 24, 2013, which is hereby incorporated by reference herein in its entirety.

What is claimed is:

1. A laser comprising:
an upper reflecting mirror;
a lower reflecting mirror; and
an active layer located between those mirrors,
wherein a plurality of individual light receiving portions is located in an optical path inside a cavity consisting of the upper reflecting mirror and the lower reflecting mirror in order to detect a laser beam.

2. The laser according to claim 1, comprising:
a first cladding layer located between the active layer and the lower reflecting mirror;
a second cladding layer located between the upper reflecting mirror and the active layer;
a gap provided in the optical path between the upper reflecting mirror and the lower reflecting mirror; and
a moving unit configured to change a distance between the lower reflecting mirror and the upper reflecting mirror.

3. The laser according to claim 1, comprising:
a first cladding layer located between the active layer and the lower reflecting mirror; and
a second cladding layer located between the upper reflecting mirror and the active layer,
wherein at least one of the plurality of light receiving portions is located on the second cladding layer.

4. The laser according to claim 1, wherein at least one of the plurality of light receiving portions is located on the upper reflecting mirror.

5. The laser according to claim 1, wherein at least one of the plurality of light receiving portions is disposed at a position in which light intensity becomes 1/10 or less of a peak value of light intensity of light to be emitted from the surface emitting laser.

6. The laser according to claim 1, wherein at least one of the plurality of light receiving portions is located at a position in which light intensity becomes 1/100 or less of a peak value of light intensity of light to be emitted from the surface emitting laser.

7. The laser according to claim 1, wherein at least one of the plurality of light receiving portions is located inside a region having a radius of 10 μm from the center of the optical path.

8. The laser according to claim 1, wherein at least one of the plurality of light receiving portions is located outside a region having a radius of 2 μm from the center of the optical path.

9. The laser according to claim 1, comprising:
a first cladding layer consisting of a semiconductor layer and located between the active layer and the lower reflecting mirror; and
a second cladding layer consisting of a semiconductor layer and located between the upper reflecting mirror and the active layer,
wherein at least one of the plurality of light receiving portions is adjacent to the first cladding layer or the second cladding layer, and consists of a semiconductor layer having a conductivity type different from that of the adjacent first cladding layer or second cladding layer.

10. The laser according to any one of claims 1, wherein the plurality of light receiving portions is connected to a circuit configured to detect a transverse mode state of a laser beam.

11. The laser according to claim 1, wherein the plurality of light receiving portions is connected to a circuit configured to measure intensity of a laser beam.

12. The laser according to claim 1, wherein a feedback loop is formed in order to feed back a signal detected by the plurality of light receiving portions to driving of the surface emitting laser.

13. The laser according to claim 1, wherein at least one of the plurality of light receiving portions is located above a current confinement layer.

14. The laser according to claim 1, wherein the number of the light receiving portions is three or more and those light receiving portions are two-dimensionally located on a surface which intersects with a radiation direction of a laser beam.

15. The laser according to claim 1, comprising:
a control unit configured to control a transverse mode state of the surface emitting laser based on light intensity detected by the plurality of light receiving portions.

16. The laser according to claim 15, wherein the control unit controls the transverse mode state of light to be a single-mode, the light being emitted from the surface emitting laser.

17. An optical apparatus comprising:
an optical source portion configured to change a wavelength of light;
an optical system configured to branch the light from the optical source portion into irradiation light to be emitted to an object and reference light, and to cause coherence light from reflected light of the light emitted to the object and from the reference light;
a light detecting unit configured to receive the coherence light; and
an information acquisition unit configured to process a signal from the light detecting unit and to acquire information of the object,
wherein the optical source portion is the laser according to claim 1.

18. The optical apparatus according to claim 17, wherein the information acquisition unit acquires information relating to a tomographic image of an organism.

19. The optical apparatus according to claim 18, wherein the information relating to the tomographic image of the organism is information relating to a tomographic image of the fundus of an eye.
